(19) United States
(12) Patent Application Publication
(10) Pub. No.: US 2009/0320855 A1
(43) Pub. Date: Dec. 31, 2009

(54) Kit for Contraception and Prevention of Sexually Transmitted Infections

(75) Inventor: Alfred A. Shihata, Del Mar, CA (US)

Correspondence Address: CATALYST LAW GROUP, APC, 9710 SCRANTON ROAD, SUITE S-170, SAN DIEGO, CA, 92121, US

(73) Assignee: Instead Sciences, Inc., San Diego, CA (US)

(21) Appl. No.: 12/553,226

(22) Filed: Sep. 3, 2009

Related U.S. Application Data
(63) Continuation of application No. 11/564,793, filed on Nov. 29, 2006, now abandoned.

(51) Int. Cl. A61F 6/06; A61M 1/00

(52) U.S. Cl. 128/830; 604/33

(57) ABSTRACT

The present invention relates to a kit for the prevention of conception and infection by sexually transmitted infections, said kit comprising the combination of a barrier-type device, a vaginal applicator, and a prophylactic agent. The present invention also relates to a barrier-type device for the prevention of conception and infection by sexually transmitted infections. The present invention also relates to a vaginal applicator useful for depositing a prophylactic agent in divided doses and in multiple directions over the barrier-type device and over a desired area of the cervix and the vagina.

### PRIORITY INFORMATION

The application is a continuation application of U.S. application Ser. No. 11/564,793 filed on Nov. 29, 2006, now pending. The disclosure of the prior application is considered part of and is incorporated by reference in the disclosure of this application.

### CROSS REFERENCES

The application is related to PCT Application Serial No. PCT/US07/85925, which also claims the benefit of priority from U.S. Provisional application Ser. No. 11/564,793 by Alfred A. Shihata, entitled “A Kit for Contraception and Prevention of Sexually Transmitted Infections,” filed on Nov. 29, 2006, and incorporated herein in its entirety by this reference.

### FIELD OF THE INVENTION

The present invention relates to the field of contraception and prevention of sexually transmitted infections. In particular, the present invention relates to a kit for the prevention of conception and infection by sexually transmitted infections, said kit comprising the combination of a barrier-type device, a vaginal applicator, and a prophylactic agent. The present invention also relates to a barrier-type device for the prevention of conception and infection by sexually transmitted infections. The present invention also relates to a vaginal applicator useful for depositing a prophylactic agent over the barrier-type device and over a desired area of the vagina and the cervix.

### BACKGROUND OF THE INVENTION

The prior art devices for intravaginal, barrier-type prevention of conception and sexually transmitted infections include the diaphragm and the cervical cap. The diaphragm, when positioned intravaginally, acts as a mechanical barrier to prevent sperm from entering the os of the cervix. A spermicide is typically used on the bowl of the diaphragm facing the cervix to kill sperm traversing over the edge of the diaphragm. Typically, the diaphragm is a shallow rubber cup with a rim. A round, coiled metal spring is disposed in the rim. The diaphragm is intended for intravaginal position between the posterior aspect of the pubic bone and the posterior fornix. When such positioned, the diaphragm presses against the vaginal wall to form a continuous mechanical barrier therewith.

The cervical cap is a small, vaulted or domed device, typically scaled to fit over a cervix, where it is retained in place by suction. In contrast, the diaphragm is retained in place by spring tension. The cervical cap includes a dome which rises from a rim. The cap is slipped over the cervical protrusion to block access to the uterus through the cervical os.

Unfortunately, traditional diaphragm and cervical cap devices are not adapted to seal closely the space between the vagina and the cervix. The diaphragm is held against the upper part of the vaginal canal by tension of the spring in its rim, while the cervical cap typically engages only the tip of the cervix. Both of these prior art devices may be dislodged by a variety of factors, such as sexual arousal, coital activity, or orgasm. It is established that dislodgment of the device reduces the effectiveness of the barrier and permits unintended conception or infection.

In failing to take into account the anatomy and physiology of the vagina and the cervix, the prior art devices form imperfect barriers against the penetration of sperm and agents of sexually transmitted infections into the uterus. It is well known that fertilization and infection take place within the uterus; therefore a more effective barrier blocking access to the cervical os provides greater protection from unwanted fertilization or infection following sexual intercourse.

With the emergence of the AIDS epidemic, there is a compelling need for intravaginal, barrier-type devices which are capable of decreasing the risk of infection by significantly increasing the effectiveness and reliability of the barrier to entry of bodily fluids as such as semen into the cervix.

U.S. Pat. No. 4,858,624 discloses a barrier-type device which is intended to be placed over the cervix to block entry of bodily fluids into the cervix. While an improvement over the prior art barrier-type devices, the device is intended to be positioned in the vagina by means of a special inserter. Such positioning is cumbersome and requires the use of a special inserter. Moreover, since the device lacks any internal removal means, its removal presents an additional difficulty to the user. The user has to manually reach inside the vagina and attempt to remove the device by applying pressure on the rim of the device in order to break the suction between the device and the cervix. Such manual removal of the device presents risk of physically injuring the interior of the vagina by finger nail scratching.

U.S. Pat. No. 5,207,232 discloses a kit for intravaginal, barrier-type prevention of conception and sexually transmitted infection, said kit comprising a barrier-type device and an inserter. While an improvement over the prior art barrier-type devices, the device suffers from many of the same problems shared by the device disclosed in U.S. Pat. No. 4,858,624. Specifically, it is cumbersome to insert and remove, presence of the inserter adds a risk of injury to the vagina, and manual removal of the device presents an additional risk of injury to the vagina.

It is one objective of the present invention to provide an intravaginal, barrier type device which provides for a more perfect and reliable coverage of the cervix intended to prevent the entry of sperms and infectious agents from the vaginal canal through the cervical os into the uterus. It is another objective of the present invention to provide an intravaginal, barrier-type device which can be easily inserted and removed manually, without the need of any additional inserter, and without an increased risk of vagina injury.

It is well known in the art of contraception that spermicidal compositions are useful to protect against pregnancy. They can be typically used alone, with a mechanical barrier-type device such as a diaphragm, cervical cap, or sponge, or an in addition to other contraceptive methods. However, unfortunately, spermicidal compositions such as nonoxynol-9 also have a significant side effect in that they destroy the natural and safe biological environment of the cervix and vagina, often resulting in cervical and vaginal damage, which in turn increases the woman's susceptibility to sexually transmitted infections.

It is desirable to use the spermicidal compositions in conjunction with intravaginal barrier-type devices to increase the success of preventing conception and sexually transmitted infection. One common problem associated with prior art devices is that spermicidal compositions applied onto or into the bowl of the device facing the cervix cause irritation to the cervix. Furthermore, the spermicidal composition originally deposited on the device first leaks outside the vagina, where it causes vaginal irritation, and then ultimately becomes ejected from the vagina, decreasing the contraceptive and anti-infectious efficacy of the spermicide. It is one objective of the present invention to provide an intravaginal barrier-type device that holds the deposited spermicide in place and prevents it from leaking and irritating the vagina.

Spermicidal compositions are typically deposited directly into the vagina or onto the intravaginal barrier-type device by means of an applicator. Prior art applicators generally consist of a reservoir having a proximal end and a distal end, a plunger or other ejection means attached to the proximal end, and a tip attached to the distal end. The reservoir is generally filled from the distal end. The spermicidal composition is generally deposited to a target area by applying pressure to the plunger or other ejection means attached to the proximal end, such that the spermicidal composition inside the reservoir travels toward the tip attached to the distal end and finally is ejected from the tip into the target area. One problem common to prior art applicators is that the spermicidal composition is ejected from a single opening in the tip attached to the distal end of the reservoir. Therefore, the ejected spermicide travels in one path and covers a relatively limited area. It is desirable to spread the spermicide or microbicide as widely as possible throughout the desired target area in order to provide a maximum amount of protection against conception and sexually transmitted infection. Users of prior art applicators typically attempt to augment this problem by depositing additional doses of spermicide or microbicide. This method is undesirable because the spermicide or microbicide is deposited in a relatively limited area and soon will be expelled from the vagina, thus increasing the chance of vaginal irritation due to the spermicide spillage outside of the vagina.

Therefore, it is a further objective of the present invention to provide a vaginal applicator that ejects spermicidal or microbicidal compositions in divided doses and in multiple directions, and thus deposits a single dose of spermicide over a wider area than prior art applicators. Yet further objectives of the present invention will become apparent from a consideration of the following description.

### SUMMARY OF THE INVENTION

In one aspect, the present invention is directed to an intravaginal barrier-type device for prevention of conception and sexually transmitted infection. In another aspect, the present invention is directed to an intravaginal barrier-type device that covers the cervix. In another aspect, the present invention is directed to an intravaginal barrier type-device for prevention of conception and sexually transmitted infection, such device comprising a cervical dome fabricated from a flexible material, said dome having an upwardly facing opening and a curved surface extending from said opening to a downwardly facing dome peak, a continuous, generally annular fornical rim defining said opening, said rim being folded from said dome inwardly, upwardly, and outwardly, transitioning therefrom into a continuous, annular vaginal brim which circumscribes said cervical dome and extends both outwardly and downwardly, said fornical rim thereby forming an inwardly extending lip and a gel trapping, inwardly extending groove between said rim and said lip, and a strap extending across the dome over the downwardly facing dome peak.

In another aspect, the present invention is directed to a vaginal applicator for depositing a prophylactic agent onto a desired target area. In one aspect of the present invention, the desired target area is the exterior surface of the intravaginal barrier-type device. In another aspect, the desired target area is the cervix and the vagina. In yet another aspect, the vaginal applicator comprises a reservoir for holding the prophylactic agent, said reservoir having a proximal end and a distal end, a perforated tip attached to the distal end, and plunger means attached to the proximal end.

In yet another aspect, the present invention is directed to a kit for prevention of conception and sexually transmitted infection. In one aspect, the kit comprises a combination of a barrier-type device for prevention of conception and sexually transmitted infection, a vaginal applicator, and a prophylactic agent. In another aspect, the kit comprises a combination of (a) a barrier-type device for prevention of conception and sexually transmitted infection, comprising a cervical dome fabricated from a flexible material, said dome having an upwardly facing opening and a curved surface extending from said opening to a downwardly facing dome peak, and a continuous, generally annular fornical rim defining said opening, said rim being folded from said dome inwardly, upwardly, and outwardly, transitioning therefrom into a continuous, annular vaginal brim which circumscribes said cervical dome and extends both outwardly and downwardly, said fornical rim thereby forming an inwardly extending lip and a gel trapping, inwardly extending groove between said brim and said lip, and a strap extending across the dome over the downwardly facing dome peak; and (b) a vaginal applicator for depositing a prophylactic agent over a desired area of the cervix and the vagina, comprising a reservoir for holding the prophylactic agent, said reservoir having a proximal end and a distal end, a perforated tip attached to the distal end, and plunger means attached to the proximal end; and (c) a prophylactic agent for deposition onto a desired target area.

These and other features, aspects, and advantages of the present invention will become better understood with reference to the following description and claims.

### BRIEF DESCRIPTION OF THE DRAWINGS

The present invention will become better understood with reference to the specification, appended claims, and accompanying drawings, where:

### NUMBERING REFERENCE LIST

10. Cervix
12. Rim
14. Femcap
16. Brim
18. Dome
20. Removal strap
22. Vagina
24. Bowl
6. Cross-section of the Femcap at level
26. Groove
28. Lip
30A. Vaginal applicator embodiment A
30B. Vaginal applicator embodiment B
32. Handle of the barrel
34. Barrel
36. Tip of the barrel
38. Oval perforations
40. Circular perforations
42. Volume capacity in centimeters
44. Speed bumps
46. Cavity of the barrel
48. Female thread to be threaded on the male thread of the microbicide tube
56. Handle of the plunger
58. Shaft of the plunger
60. Tip of the plunger
62. Raised ledge of the plunger tip
50. Disposable tip
52. Thread of the disposable tip

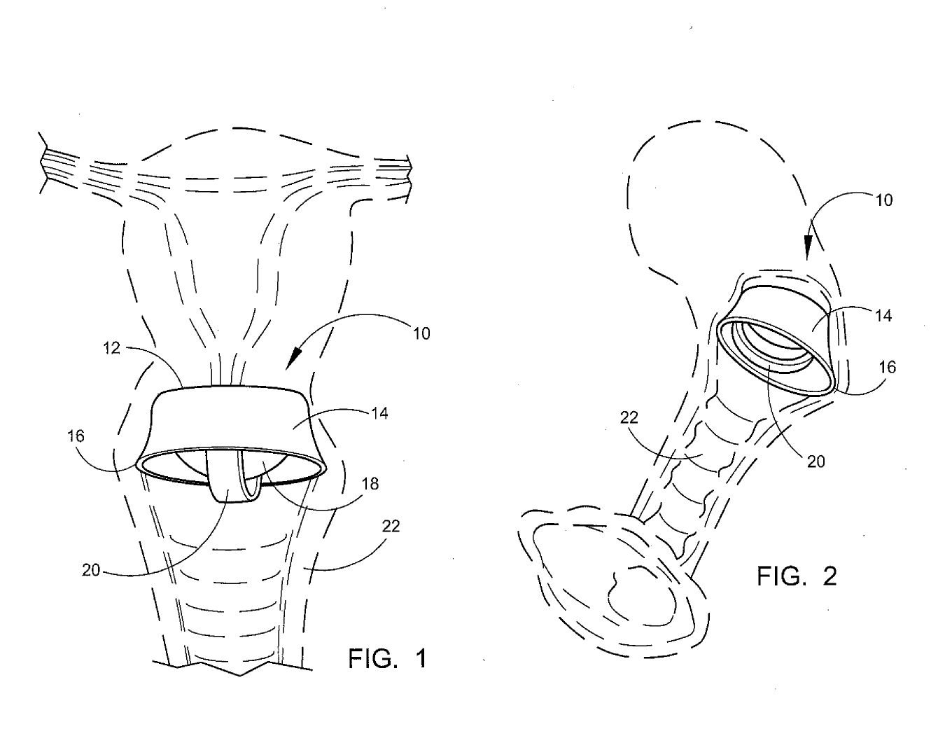

FIG. 1 shows a front view of the uterus and cervix covered by the intravaginal barrier-type device.

FIG. 2 shows a lateral view of the uterus and cervix covered by the Femcap. Cervix 10 is covered by the Femcap and not shown.

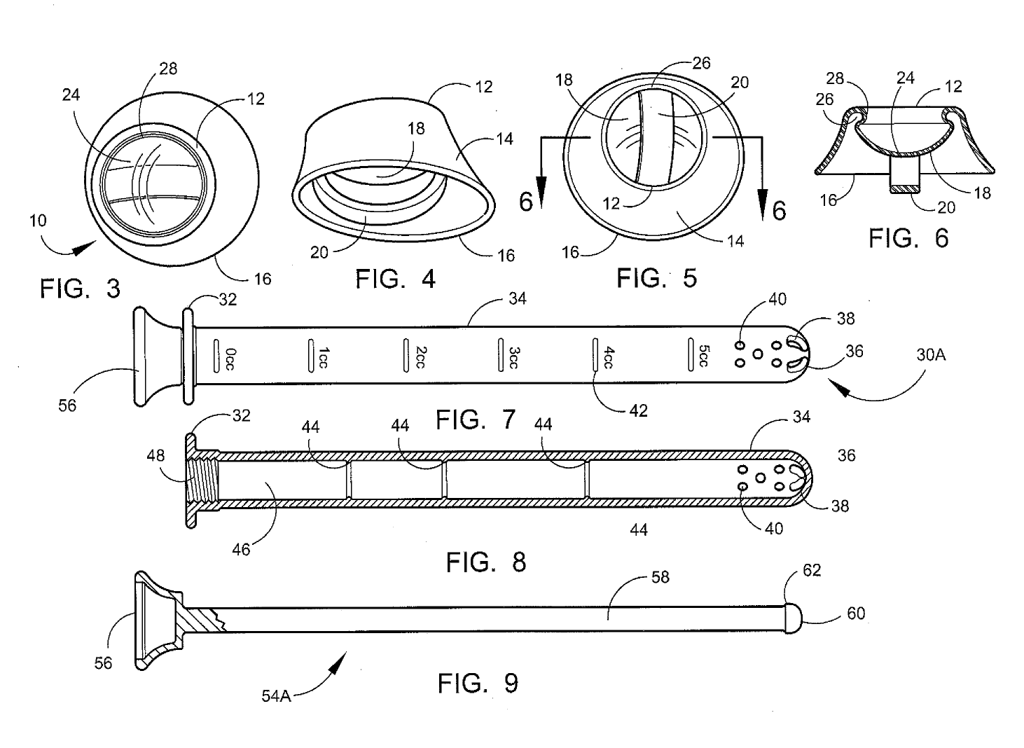

FIG. 3 shows a cervical view of the Femcap.

FIG. 4 shows a lateral view of the Femcap.

FIG. 5 shows a vaginal view of the Femcap.

FIG. 6 shows a cross section view of the Femcap at a level view.

FIG. 7 shows Embodiment A of the vaginal applicator.

FIG. 8 shows a barrel of the vaginal applicator.

FIG. 9 shows a plunger of the vaginal applicator.

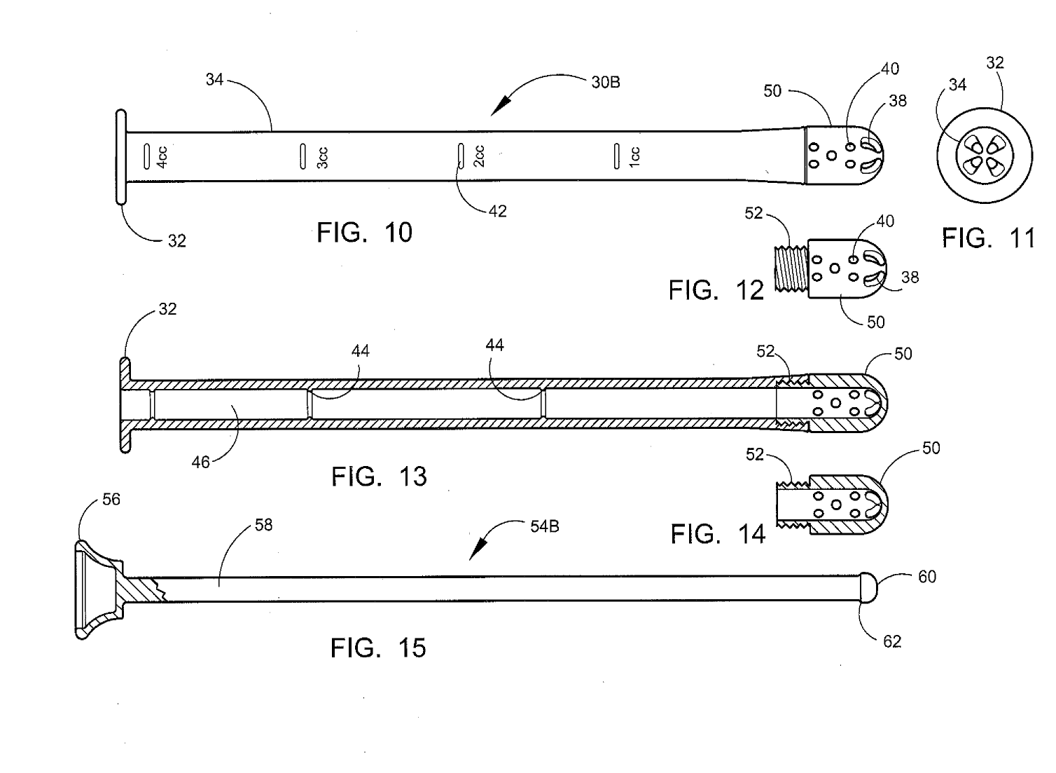

FIG. 10 shows the barrel of Embodiment B of the vaginal applicator.

FIG. 11 shows a top view of the disposable tip.

FIG. 12 shows a side view of the disposable tip.

FIG. 13 shows the disposable tip threaded to the barrel of the vaginal applicator.

FIG. 14 shows another embodiment of the disposable tip.

FIG. 15 shows another embodiment of the plunger.

### DESCRIPTION

One embodiment of the present invention, referred to herein as embodiment 1, is a kit for prevention of conception and sexually transmitted infection, comprising a combination of a barrier-type device, a vaginal applicator for depositing a prophylactic agent onto a desired target area, and a prophylactic agent. In a preferred embodiment of the present invention, the barrier-type device fits over the cervix and blocks entry of sperm and infectious agents from the vaginal canal through the cervical os into the uterus. FIGS. 1 and 2 illustrate the intravaginal, barrier-type device of the present invention covering the cervix to prevent entry of sperm and infectious agents.

In the preferred embodiment of the present invention, referred to herein as embodiment 2, the barrier-type device of embodiment 1 comprises a cervical dome fabricated from a flexible material, said dome having an upwardly facing opening and a curved surface extending from said opening to a downwardly facing dome peak, and a continuous, generally annular fornical rim defining said opening, said rim being folded from said dome inwardly, upwardly, and outwardly, transitioning therefrom into a continuous, annular vaginal brim which circumscribes said cervical dome and extends both outwardly and downwardly, said fornical rim thereby forming an inwardly extending lip and a gel trapping, inwardly extending groove between said brim and said lip, and a strap extending across the dome over the downwardly facing dome peak.

FIGS. 3, 4, 5 and 6 illustrate an intravaginal, barrier-type device of the present invention comprising bowl 24, rim 12, brim 16, dome 18, groove 26, strap 20, and lip 28. Preferably, the device is a single piece formed by molding a flexible, physiologically non-reactive material, such as silicone rubber. The cervical dome 18 preferably has a hemi-spherical or hemi-ovoid shape forming a cervical enclosure. At the opening of the enclosure, the dome 18 transitions to a fornical rim 12, which in turn, transitions by a backward fold to a vaginal brim 16. An annular lip 28 is formed in the intravaginal, barrier-type device of the present invention by an inward extension of the rim 12 along the periphery of the opening into the dome 18. The lip 28 juts into the opening toward the center line of the dome 18. The backward fold of the rim 12 at the lip 28 by which the rim 12 transitions to the brim 16 forms an annular groove 26 between the brim 16 and the dome 18. Strap 20 extends across the dome 18, over the downwardly facing dome peak. Strap 20 provides a convenient finger grip for manual removal of the intravaginal, barrier-type device of the present invention. Preferably, the strap 20 is fabricated from the same flexible, physiologically non-reactive material as the device.

The relationship of the structure of the barrier-type device of embodiment 2 to female anatomy is illustrated in FIGS. 1 and 2. As shown, the barrier-type device covers the cervix entirely, such that the cervical dome 18 engages the cervix, which extends through the opening defined by the rim 12. The rim 12 is seated at the bottom of the fornices. The brim 16 extends from the bottom of the fornices in close sealing engagement with the vaginal walls, therefore continuing a circumferential seal along the vagina forwardly from the fornices. Preferably, the barrier-type device is positioned over the cervix in such way as to orient the annular groove 26 toward the opening of the vagina. Thus oriented, the groove 26 will trap fluids traveling along the inside of the brim 16 toward the dome 18. The strap 20 extends across the dome 18 over the downwardly facing dome peak. In the preferred embodiment, strap 20 is used to manually pull on and remove the barrier-type device from the cervix.

When used in conjunction with a prophylactic agent, the intravaginal barrier-type device of the present invention increases the effectiveness of the prophylactic agent by spreading it uniformly all over the cervical and vaginal surfaces in contact with the barrier-type device. In the preferred embodiment, prophylactic agent is deposited into the storage groove 26 and onto the device surfaces in contact with vaginal walls and cervical area. When filled with a prophylactic agent, the groove 26 provides a large surface area which traps and kills sperm and pathogenic organisms upon contact as soon as they enter the vagina.

In another embodiment of the present invention, referred to herein as embodiment 3, the vaginal applicator of embodiment 1 comprises a reservoir for holding a prophylactic agent, said reservoir having a proximal end and a distal end, and a perforated tip attached to the distal end, and plunger means attached to the proximal end. FIG. 7 shows a preferred embodiment of the vaginal applicator of the present invention, where a barrel 34 having a proximal end and a distal end is connected to a perforated tip 36 at the distal end, and plunger means 56 at the proximal end. The vaginal applicator 30A is used to deposit a prophylactic agent to a desired target area. Preferably the prophylactic agent is filled into the barrel 34, and is ejected from the barrel 34 through the perforated tip 36 by plunger means 56.

FIG. 11 shows a close-up of the perforated tip 36 of the vaginal applicator of the present invention, where the tip comprises multiple oval perforations 38 on its surface. The number, size, shape, and location of the perforations may vary. Such variations will be apparent to one of reasonable skill in the art and are within the scope of the present invention. In the preferred embodiment, a single dose of a prophylactic agent ejected through the perforated tip is dispersed through the multiple perforations on the tip. As a result of the multiple perforations, a dose of a prophylactic agent ejected through the perforated tip of the present invention covers a wider desired target area than the same dose ejected through a conventional tip comprising a single hole.

In one alternative embodiment, the barrel of the vaginal applicator of embodiment 3 is filled with the prophylactic agent from the proximal end. In another alternative embodiment, the reservoir of the vaginal applicator of embodiment 3 is filled with the prophylactic agent from the distal end. In another alternative embodiment, the vaginal applicator of embodiment 3 is single use. In another alternative embodiment, the vaginal applicator of embodiment 3 is reusable. These and other alternatives are well within the spirit and scope of the present invention.

Another embodiment of the present invention, referred to herein as embodiment 4, is the vaginal applicator of embodiment 3, wherein the entire contents of the reservoir are discharged through the perforated tip in a single action of the plunger means. For example, in such embodiment the user has to press the plunger means once for the entire dose of the prophylactic agent held in the reservoir to be ejected through the perforated tip onto the desired target area. In this embodiment, if for example the reservoir maximum capacity is 3 grams and 3 grams of prophylactic agent are contained in the reservoir, a single operation of the plunger means will cause the entire 3 grams dose to be ejected through the perforated tip and onto the desired target area. In one embodiment of the present invention, the vaginal applicator of embodiment 4 is filled with the prophylactic agent from the proximal end. In an alternative embodiment, the vaginal applicator of embodiment 4 is filled from the distal end. In one embodiment, the vaginal applicator of embodiment 4 is single use. In another alternative embodiment, the vaginal applicator of embodiment 4 is reusable. These and other alternatives are well within the spirit and scope of the present invention.

An alternative embodiment of the present invention, referred to herein as embodiment 5, is the vaginal applicator of embodiment 3, wherein a pre-determined portion of the entire contents of the reservoir is discharged through the perforated tip in divided doses by multiple actions of the plunger means. For example, in such embodiment the user has to press the plunger more than once in order to the entire dose of prophylactic agent held in the reservoir to be ejected through the perforated tip onto the desired target areas. In this embodiment, if for example the reservoir maximum capacity is 3 grams, the reservoir is filled to capacity, and the vaginal applicator is set to eject 1 gram with each action of the plunger means, then a single operation of the plunger means will cause a 1 gram dose to be ejected through the perforated tip and onto the desired target area. In one embodiment of the present invention, the vaginal applicator of embodiment 5 is filled with the prophylactic agent from the proximal end. In an alternative embodiment, the vaginal applicator of embodiment 5 is filled from the distal end. In one embodiment, the vaginal applicator of embodiment 5 is single use. In another alternative embodiment, the vaginal applicator of embodiment 5 is reusable. These and other alternatives are well within the spirit and scope of the present invention.

The kit of the present invention may be used in conjunction with a variety of prophylactic agents currently on the market, including, but not limited to spermicides, microbicides, and virucides. The prophylactic agent used in conjunction with the kit of the present invention may take the form of cream, gel, jelly, or foam. In the preferred embodiment, the kit of the present invention is used in conjunction with Acidform (gel, available from TOPCAD Chicago, Ill.). Alternatively, the kit of the present invention may be used in conjunction with various additional spermicidal and microbicidal products not limited to Acidform gel. Alternatively, the kit of the present invention may be used with a prophylactic agent that is a combination of a spermicide and a peroxygen compound. In one embodiment, the kit of the present invention is used in conjunction with a spermicide or microbicide. Practitioners of reasonable skill in the art will be able to choose appropriate prophylactic agents for use in conjunction with the kit of the present invention.

In the preferred embodiment of the present invention, the prophylactic agent is applied over the interior and exterior surfaces of the intravaginal barrier-type device. In another embodiment, the prophylactic agent is applied both over the surfaces of the barrier-type device and directly over a desired target area of the cervix and the vagina. In one embodiment, the prophylactic agent is applied onto the cervix and the vagina.

While this invention has been particularly shown and described with references to preferred embodiments thereof, it will be understood by those skilled in the art that various changes in form and details may be made therein without departing from the scope of the invention encompassed by the appended claims.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings failing within the scope of the generic disclosure also form part of these inventions. This includes the generic description of each invention with a proviso or negative limitation removing any subject matter from the genus, regardless of whether or not the excised materials specifically resided therein.

### REFERENCES

The following references are cited in the specification; all of these references are incorporated by this reference in the application in their entirety:
1. U.S. Pat. No. 4,858,624 (1989) Shihata
2. U.S. Pat. No. 4,989,618 (1991) Shihata
3. U.S. Pat. No. 5,207,232 (1993) Shihata
4. U.S. Pat. No. 5,778,886 (1998) Shihata
5. U.S. Pat. No. 6,706,276 (2004) Garg, et al.
6. U.S. Pat. No. 6,239,182 (2001) Zaneveld et al.
7. U.S. Pat. No. 6,125,850 (2000) Sokal et al.
8. U.S. Pat. No. 6,028,115 (2000) Zaneveld et al.

I claim:

1. A vaginal applicator for depositing a prophylactic agent over an entire circumference and length of the vagina, comprising:
a reservoir for holding the prophylactic agent, the reservoir having a proximal end and a distal end;
a tip having a plurality of perforations attached to the distal end, the plurality of perforations providing a wide target area; and
a plunger having a plunger proximal end and a plunger distal end, the plunger proximal end having a mating portion to a woman's finger or thumb, the mating portion having a smaller diameter gradually increasing to a larger diameter at the plunger proximal end.

2. The applicator of claim 1, wherein the reservoir is filled with the prophylactic agent from its proximal end, and wherein the plurality of perforations comprises oval perforations and circular perforations of varying dimensions to eject the prophylactic agent at different forces for a wider distribution.

3. The applicator of claim 1, wherein the reservoir is filled with the prophylactic agent from its distal end, and wherein the reservoir comprises a plurality of volume indicators, the plurality of volume indicators increasing with respect from the distal end to the proximal end.

4. The applicator of claim 3, wherein the plurality of volume indicators are in cubic centimeters (cc).

5. The applicator of claim 1, wherein the perforated tip is removably attached to the distal end.

6. The applicator of claim 1, wherein the reservoir further comprises:
female threads at the proximal end thereof, the female thread configured to receive male threads of a microbicide tube for filling the reservoir with microbicide; and
a plurality of volume indicators increasing with respect from the proximal end to the distal end.

7. The applicator of claim 1 further comprising bumps, wherein the bumps provide for the prophylactic agent to be deposited into the entire circumference of the vagina in divided doses, wherein the divided doses comprise approximately one (1) gram each of the agent.

8. A kit for prevention of conception and sexually transmitted infection, comprising, in combination:
a barrier-type device for prevention of conception and sexually transmitted infection, comprising:
a cervical dome fabricated from a flexible material, the dome having an upwardly facing opening and a curved surface extending from the opening to a downwardly facing dome peak;
a continuous, generally annular fornical rim defining the opening, the rim being folded from the dome inwardly, upwardly, and outwardly, transitioning therefrom into a continuous, annular vaginal brim which circumscribes the cervical dome and extends both outwardly and downwardly, the fornical rim thereby forming an inwardly extending lip and a gel trapping, inwardly extending groove between the brim and the lip;
a removal strap extending across the dome over the downwardly facing dome peak; and
a vaginal applicator for depositing a prophylactic agent over a desired area of the vagina, comprising:
a reservoir for holding the prophylactic agent, the reservoir having a proximal end and a distal end;
a plurality of volume indicators along a length of the reservoir;
a perforated tip attached to the distal end;
plunger means attached to the proximal end; and
a prophylactic agent for deposition over a desired area of the vagina.

9. The kit of claim 8, wherein the prophylactic agent is selected from a group consisting of: a spermicide, a microbicide, and a virucide.

10. The kit of claim 9, wherein the spermicide or microbicide is Acidform.

11. The kit of claim 8, wherein the prophylactic agent is a combination of a spermicide and a peroxygen compound.

12. The kit of claim 11, wherein the peroxygen compound is hydrogen peroxide.

13. The kit of claim 8, wherein the prophylactic agent is in the form selected from the group consisting of: a cream, a gel, a jelly, and a foam.

14. The kit of claim 8, wherein the prophylactic agent is deposited over a desired area of the vagina, the area selected from the group consisting of: cervix and vaginal walls.

15. The kit of claim 8, the vaginal applicator further comprising a barrel having bumps, wherein the bumps provide for the prophylactic agent to be deposited into an entire circumference and length of the vagina in divided doses, and wherein the plunger means comprises a bulbous tip, the bulbous tip being continuously smooth to form a raised ledge for mating with the bumps to provide the divided doses.

16. A method of contraception and preventing sexually transmitted diseases comprising:
inserting a barrier-type device into a vagina to cover a cervix, the barrier-type device having a strap assisting the inserting;
providing female threads to a proximal end of a reservoir of a vaginal applicator, the vaginal applicator further having a plunger;
mating the female threads to a threaded portion of a microbicide tube;
squeezing the microbicide tube to fill the reservoir;
indicating a plurality of volumes within the reservoir;
perforating a distal end of the reservoir with oval and circular perforations;
varying a plurality of dimensions of the oval and circular perforations;
providing a bulbous tip to a distal end of the plunger, the bulbous tip continuously rounded to form a raised ledge;
inserting the vaginal applicator;
engaging the plunger to apply the microbicide to an external side of the barrier type device;
re-engaging the plunger to apply the microbicide to an entire length of the vagina in measured doses;
ejecting the microbicide at different forces for wider distribution provided by the varying the plurality of dimensions; and
combining an efficacy of the barrier-type device with an efficacy of an application of the microbicide to prevent pregnancy.

17. The method of contraception and preventing sexually transmitted diseases of claim 16, further comprising:
providing bumps to the reservoir; and
assisting the measured doses with the bumps.

18. The method of contraception and preventing sexually transmitted diseases of claim 17, further comprising providing a mating portion to a proximal end of the plunger, the mating portion mating to a woman's finger or thumb, the mating portion having a smaller diameter gradually increasing to a larger diameter at the plunger proximal end.
